(19) United States
(12) Patent Application Publication
(10) Pub. No.: US 2005/0042182 A1
(43) Pub. Date: Feb. 24, 2005

(54) Topical compositions of urea

(76) Inventors: Moshe Arkin, Kfar-Shemaryahu (IL); Nir Avram, Meitar (IL); Olga Buriakovsky, Beer Sheva (IL); Amira Zeevi, Omer (IL); Benjamin Schneider, Rehovot (IL); Stephen Cherkez, Caesarea (IL)

Correspondence Address: Martin Moynihan;c/o ANTHONY CASTORINA, SUITE 207, 2001 JEFFERSON DAVIS HIGHWAY, ARLINGTON, VA, 22202, US

(21) Appl. No.: 10/850,435

(22) Filed: May 21, 2004

Related U.S. Application Data
(60) Provisional application No. 60/494,579, filed on Aug. 13, 2003.
(60) Provisional application No. 60/494,581, filed on Aug. 13, 2003.
(60) Provisional application No. 60/510,554, filed on Oct. 14, 2003.
(60) Provisional application No. 60/527,279, filed on Dec. 8, 2003.

(51) Int. Cl. A61K 9/00

(52) U.S. Cl. 424/47

(57) ABSTRACT

Pharmaceutical, cosmetic and cosmeceutical foamable compositions for topical application, containing, as an active ingredient, urea and/or a derivative thereof, processes of manufacturing these compositions and uses of these compositions in the treatment of various dermatological conditions such as, for example, conditions associated with dry skin and/or scalp.

This application claims the benefit of priority from U.S. Provisional Patent Application Nos. 60/494,579, 60/494,581, filed Aug. 13, 2003, U.S. Provisional Patent Application No. 60/510,554, filed Oct. 14, 2003 and U.S. Provisional Patent Application No. 60/527,279, filed Dec. 8, 2003, the teachings of which are hereby incorporated by reference in their entirety.

### FIELD AND BACKGROUND OF THE INVENTION

The present invention relates to pharmaceutical, cosmetic and cosmeceutical compositions for topical application, and their use in the treatment of medical, cosmetic and cosmeceutical conditions such as dry skin and/or scalp.

Dry skin is a common condition associated with a plurality of disorders and frequently requires therapeutic intervention.

Dermatologists often cali dry skin in later life “xerosis” or “ichthyosis”. Xerosis is a term used to describe abnormal skin dryness. Ichthyosis is a term used to describe a group of cutaneous disorders characterized by increased or aberrant keratinisation, and resulting in non-inflammatory scaling of the skin. There are at least twenty varieties of ichthyosis, including inherited and acquired forms. Further details regarding xerosis and ichthyosis can be found in “Atlas of Clinical Dermatology” by Anthony du Vivier, 3rd edition (Jul. 17, 2002) Publisher: Churchill Livingstone, which is incorporated herein by reference.

Dry skin often leads to dermatitis, a condition in which the skin becomes red and itchy, and which is typically characterized by a crazy-paving appearance on the lower legs (eczema craquele) or round patches scattered over the trunk and limbs (a dry form of nummular dermatitis). In some cases of dermatitis, such as, for example, winter itch, 7th age itch, or senile pruritus, the dry skin is just itchy, without much of a rash.

Dry skin results from, or is aggravated by, low humidity, sunlight, abrasive clothing and/or a repeated use of soaps, detergents or other lipid solvents, and is further strongly influenced by factors such as age, race, genetics, climate and lifestyle.

Numerous humidifying topical preparations containing emollients and moisturizers have been used over the years in the treatment of dry skin and more acute dermatological disorders which exhibit dry skin symptoms, such as, for example, ichthyosis, psoriasis, actinic damage, eczema and the like.

As is known in the art, the terms “moisturizer” (to add moisture) and “emollient” (to soften) are interchangeable as they describe different effects of the same agents on the skin, as is further detailed hereinunder.

“Moisturizers” is a general term used to describe substances that exert two basic actions: humectants, which are introduced into the stratum corneum to increase its water holding capacity; and occlusives, which provide a layer of oil on the surface of the skin to slow water loss and thus increase the moisture content of the stratum corneum. Some moisturizers contain both occlusives and humectants.

“Emollients” is a general term used to describe substances that cover the surface of the stratum corneum so as to prevent moisture loss, thus resulting in the closure of microcracks and fissures and restoration of the natural epidermal barrier. (Marie Loden, Clinics in Dermatology, 21, 145-157, 2003).

Herein, the terms “moisturizer”, “humectant”, “emollient” and the term “hydrating agent” are used interchangeably.

As is well recognized in the art, the final form of a topical composition plays an important role in its efficacy and its usage convenience, particularly in cases where the composition is used to treat a skin condition associated with dry skin and/or scalp.

The challenge in topically applying a composition is to achieve percutaneous penetration of the active agent to the site of treatment, in many cases the epidermis. At the same time, it is important that the composition should have desirable characteristics. Hence, application should be easy, smooth and should result in no irritation, discomfort or inconvenience. Desirably, the composition should not leave a residue on the surface of the skin.

Topical compositions in forms such as gels, ointments, lotions, creams, pads and pastes are often very viscous, requiring substantial rubbing to achieve penetration of the active agent to the affected skin layer, an act which often results in discomfort and further irritation. Non-viscous creams and lotions require quick and dexterous application as they are inclined to flow off the site of treatment before penetration of the active ingredient is achieved.

Contrary to the above, foams are well suited for the topical application of compositions. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system.

A foam composition has physical characteristics which are dependent, at least in part, upon the choice and relative amounts of components such as solvents, propellants and surfactants, which may be present in the composition. The combination of these components determines the stability of the foam, which may retain its foam-like structure upon application or, alternatively, may be “a slow-breaking foam” or “a quick-breaking foam”, whereby this terminology relates to the behavior of the foam towards shearing action as is sustained when the foam is rubbed into or spread over a surface onto which it has been dispensed.

Many of the physical characteristics of foam compositions render it highly beneficial and advantageous over other forms. One such exemplary characteristic is the semi-solid to solid nature of the foam matrix, which allows the composition to be applied with the hand in any orientation without the risk of run off. Another beneficial characteristic of foams is their convenient application to large areas of the body surface. Furthermore, although foams can be water-based or hydroalcoholic, typically they are formulated with high alcohol content which, upon application to the skin of a user, quickly evaporates, driving the active ingredient through the upper skin layers to the site of treatment.

Urea is one of the most well-known and widely used humectants. Urea is used in various biological systems, serving, inter alia, as a modifier of protein solubility. Urea is known to exert antibacterial activity as well as protein complexes denaturation activity. In topical applications, urea in known to act as a penetrating moisturizer with high osmotic activity, attributed to its capability to break hydrogen bonds in the outer layers of the stratum corneum, thus dispersing epidermal keratin and exposing water-binding sites. Urea also has a stabilizing effect on the stratum corneum barrier, which can be demonstrated by reduction of trans-epidermal water loss (TEWL) and of irritative hyperemia produced by the application of an irritant (John Ademola et al., Am. J. Clin. Dermatol 3(3), 217-222, 2002).

Urea-containing preparations have been efficiently used in the treatment various afflictions related to dry skin. While preparations that contain urea concentration lower than 10 weight percentages have generally been used as skin moisturizers, preparations that contain urea concentration of 10 weight percentages or higher have been used as skin remedies, treating severe cases of dry, rough skin, such as ichthyosis and psoriasis. A representative example of a commercially available family of 40% urea-containing preparations is the Carmol®40 cream, gel and lotion (marketed by Doak Dermatologics, a subsidiary of Bradley Pharmaceuticals Inc.), which is known as a tissue softener.

As early as the 1960s, topical compositions containing urea were used in the treatment of various dermatological conditions. For example, Swanbeck (in Acta derm-vener., 48, 123, 1968) reported that soaking of pieces of horny layer from normal, ichthyotic and psoriatic skin in a 30% urea solution resulted in a considerable increase in the water binding capacity of the skin, and suggested that a cream containing urea in a concentration of 10% may be used for the treatment of ichthyosis and other hyperkeratotic conditions.

Later on, Swanbeck and Rajka (Acta derm-vener 50, 225, 1970) presented a study in which solutions containing 20% urea were used in the treatment of pruritus.

In addition, Swanbeck published a review named “Urea in the treatment of dry skin”, which teaches that dry and xerotic skin of unspecified etiology can be efficiently treated with an urea-containing cream (Swanbeck G., Acta derm-vener, 177, 7-8, 1992).

Stewart et al. presented a study in which patients suffering from ichthyosis, xerosis and atrophic senile dryness of the skin were treated with a 40% urea cream (Stewart et al., Cutis, 5, 1241, 1969).

Additional studies of various topical compositions that contain high concentrations of urea, are presented, for example, in Vleeschouwer and Bersaques (Arch. Belges. Derm. Syph 27, 225, 1971), Bien and Borkowski (Przegl. Derm. 61, 351, 1974), Pegum (Brit. J. Derm. 84, 602, 1971) and Millar (J. Am. Med. Ass. 100, 1684, 1933).

Nevertheless, it is well recognized that topical preparations that contain high concentrations of urea suffer many disadvantages. For example, commercially available formulations such as the above-mentioned Carmol®40 are characterized by an alkaline pH, namely a pH value higher than 8.0. Such a pH value is much higher than that of the natural skin (about 5.5), and may therefore cause irritations when applied. Moreover, topical application of formulations that contain high urea concentrations are typically associated with an unpleasant odor of ammonia, formed by the decomposition of urea, stickiness, and white stains that remain on the skin and clothing after the evaporation of the solvent.

European Patent Application No. 0101887A2 discloses cosmetic compositions that comprise an aqueous solution of urea or derivatives thereof, in a concentration of between 0.5 M and 12 M, and an ammonium salt of an unreactive acid, which is added to adjust the pH of the solution to between 6.0 and 8.0. According to the teachings of this patent application, the ammonium salt is aimed at retarding the production of titratable alkali from the aqueous urea solution, to thereby prolong the shelf-life of the composition. Preferred ammonium salts, according to this patent application, include ammonium salts of strong acids such as carboxylic acids having up to four carbon atoms. The instability of urea in aqueous solutions is widely taught by this reference.

JP 59020217 (to Kawaken Fine Chemical KK) describes an aqueous, jelly-like composition containing between 1 and 48 weight percentages urea, an ammonium compound and a carboxyvinyl polymer. According to the teachings of this patent, the pH of the composition is adjusted to 5.5-7.5, by adding a base made up of hydroxides of alkali metals, alkanolamines, basic amino acids and aqueous ammonia. JP 59020217 further teaches that the combination of all the components present in the disclosed composition synergistically provides for inhibition of the decomposition of urea. It is therefore implied in this patent that the stability of the composition would be reduced if any of the constitutional components would be missing.

In view of its high moisturizing performance and the disadvantages associated with the presently available urea-containing topical preparations, the present inventors have envisioned that a topical composition, formulated as a foam, which comprises urea as an active ingredient, would be a highly potent composition for treating a variety of dermatological conditions, and particularly dry skin and scalp conditions and associated disorders.

WO 86/00014 (to Weiner M.) discloses a method for the prevention and/or reduction of skin damage caused by ultraviolet radiation, which is effected by topically applying a composition comprising urea and a pharmaceutically acceptable carrier. The urea is used in this composition as an agent that moderates the effect of nitrate reduction products, which are formed as a result of exposure to ultraviolet radiation. According to the teachings of this reference, the preferred concentration of urea is from about 0.1 to about 40 weight percentages, more preferably between about 0.1 and about 20 weight percentages of the total weight of the composition. Further according to the teachings of this reference, the composition is applied to the skin in the form of conventional alcoholic lotions, liquid emulsions, creams, transparent gels, or aerosol sprays. This reference therefore fails to teach a composition that is directed to treat a dry skin and/or scalp condition and is further not directed to such compositions that are foamable.

U.S. Pat. No. 5,919,470 (to Bradley Pharmaceuticals Inc.) discloses a dermatological composition that comprises from about 21 to about 40 weight percentages urea and a method of treating xerosis, which is effected by applying the composition. In a preferred embodiment of this patent, the dermatological composition further comprises skin protectants of an oleaginous nature, derived from petroleum, emulsifiers and thickeners, and is in a semi-solid form at room temperature. According to the teachings of this patent, the composition is preferably formulated as a cream. Hence, this patent fails to teach compositions that comprises less than 21 weight percentages urea and is not directed to foamable compositions.

U.S. Pat. Nos. 6,281,239, 6,429,231 and 6,495,602, and U.S. Patent Application No. 2003064969 (all to Bradley Pharmaceuticals Inc.) disclose various compositions, all containing urea in concentrations of up to 40 weight percentages, in combination with other active ingredients such as, for example, anti-fungals (U.S. Pat. No. 6,281,239), sulfacetamide and sulfur (U.S. Pat. No. 6,429,231), astringents such as calcium acetate and aluminum sulfate (U.S. Pat. No. 6,495,602) and antimicrobials (U.S. Patent Application No. 2003064969), for the treatment of various dermatological conditions. Although the compositions disclosed in these patents and patent application are not limited to a particular form, some of these references teach that the composition is preferably applied in the form of a cream and/or a lotion. These references are therefore not directed to foamable compositions for treating dry skin and/or scalp conditions.

U.S. Pat. No. 6,423,323 (to Stephanie Neubourg) discloses a foam skin cream composition that is prepared by a specific process, which can optionally contain urea, as a hydratizing agent. As urea is used as an optional adjuvant, according to the teachings of this patent, it is used in a relatively low concentration of up to 20%. The process taught by this reference includes adjusting the pH of the composition to between 7.6 and 8.2. Such a pH value is known to be highly disadvantageous in topical application, as it is substantially higher than that of the skin (pH of 5.5), as is discussed hereinabove.

JP Patent Application No. 2002-275454 (to Daizo Co., Ltd.) discloses a water-containing aerosol composition containing a stock solution and a propellant; the stock solution being an aqueous solution containing 10-50% of a nitrogen-containing water-soluble component, whereby the nitrogen-containing component is preferably urea. The nitrogen-containing component, according to the teachings of this reference, is aimed at stabilizing the aqueous stock solution by preventing its freezing, to thereby enable a perfect dispersion state of the composition while being used at a low temperature. The composition taught by this patent application is therefore not directed at treating dry skin conditions.

JP Patent Application No. 2003-12511 (to Rohto Pharmaceutical Co., Ltd) discloses an aerosol composition that comprises an aqueous stock solution of urea in a concentration of between 2 and 35 weight percentages, and between 30 and 90 weight percentages water. According to the teachings of this patent application, the compositions are aimed at stabilizing an effective amount of urea, by mixing the propellant and the stock solution at certain volumes ratio, in a liquid state. Preferred propellants are a dimethylether or liquefied petroleum gas, which are commonly used in aerosol compositions. However, although this patent teaches compositions of urea and a propellant, it fails to teach such compositions which are formulated as a foam.

U.S. Pat. No. 5,679,324 (to Procter & Gamble Co.) discloses quick-breaking foamable fragrance compositions, which comprise a surfactant, a propellant, a fragrance and a thickener. These compositions, according to the teachings of this patent, may optionally further include skin moisturizers such as urea. According to the teachings of this patent, urea, in a concentration of between 0.1% and about 10%, may further be added to the compositions as an optional medicament that may be included in the composition. As this patent is directed to fragrance compositions, it fails to teach compositions for treating dry skin conditions, in which urea is present in an effective amount for treating these conditions.

U.S. Pat. No. 6,086,903 (to Proctor & Gamble Company) discloses a personal treatment composition that comprises an enduring perfume composition. According to the teachings of this patent, urea may optionally be added to the composition as a moisturizer, in a concentration of, as arbitrarily stated, between 0.1% and 20%, preferably, as stated, in a low concentration of between 2% and 5%. Again, this reference is directed to personal treatment compositions in which urea is an optional adjuvant and fails to teach compositions for treating dry skin conditions, in which urea is present in an effective amount for treating these conditions.

U.S. Patent Application No. 20020151446 (to Playtex Products, Inc.) discloses a foaming cleanser composition that comprises a mild surfactant system, a moisturizer system and a solvent system. According to the teachings of this patent, urea may also be included in the disclosed composition, as a humectant, in an amount that ranges between about 1 weight percentages and about 5 weight percentages. Hence, the composition disclosed in this patent application comprises low concentrations of urea and is aimed at cleansing and conditioning of hair and skin. While this patent is directed to cleansing and conditioning of hair and skin, it fails to teach compositions for treating diseased or compromised skin.

Thus, the prior art fails to teach foamable compositions for treating dry skin and/or scalp conditions and associated disorders, which include urea or any analogs or derivatives thereof in an effective amount.

As the presently available urea compositions are highly disadvantageous, particularly in treating dry skin and scalp conditions, as is discussed hereinabove, and as foamable compositions are highly advantageous in this respect, there is a widely recognized need for, and it would be highly advantageous to have, foamable compositions of urea, derivatives or analogs thereof for treating dry skin and scalp conditions and related disorders, as well as other medical, cosmetic and cosmeceutical disorders, devoid of the above limitations.

### SUMMARY OF THE INVENTION

The present inventors have now surprisingly found that foamable compositions that comprise urea and/or a derivative thereof, in a relatively high concentration, can serve as efficient pharmaceutical, cosmetic and cosmeceutical compositions for the treatment of various dermatological disorders (e.g., dry skin and/or scalp).

Hence, according to one aspect of the present invention there is provided a foamable pharmaceutical, cosmetic or cosmeceutical composition for topical application, which is identified for use in the treatment of a medical, cosmetic and/or cosmeceutical condition associated with dry skin and/or scalp. The composition of the present invention comprises urea and/or a derivative thereof, one or more propellant(s) and a pharmaceutically, cosmetically or cosmeceutically acceptable carrier.

According to further features in preferred embodiments of the invention described below, the foamable pharmaceutical, cosmetic or cosmeceutical compositions of the present invention is packaged in a packaging material and identified in print, in or on the packaging material, for use in the treatment of a medical, cosmetic and/or cosmeceutical condition associated with dry skin and/or scalp, such as, but not limited to, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, and xeroderma pigmentosum.

According to still further features in the described preferred embodiments the concentration of the urea and/or the derivative thereof is greater than 5 weight percentages of the total weight of the composition. More preferably it is greater than 10 weight percentages.

According to still further features in the described preferred embodiments the concentration of the urea and/or the derivative thereof ranges between about 5.1 weight percentages and about 48 weight percentages of the composition. More preferably, it ranges between about 20 weight percentages and about 48 weight percentages of the composition.

The concentration of the propellant(s) preferably ranges between about 0.5 weight percentage and about 60 weight percentages, more preferably between about 10 weight percentages and about 20 weight percentages.

The propellant(s) preferably include propane, iso-butane, n-butane, isopentane, n-pentane, and mixtures thereof.

According to still further features in the described preferred embodiments the composition of the present invention further comprises one or more additional active ingredient(s) such as, but not limited to, an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an antihistamine, a vitamin, a hormone and an antidandruff agent. Such a composition can be further identified for use in the treatment of a condition in which applying this additional active agent is beneficial.

According to still further features in the described preferred embodiments the composition of the present invention further comprises one or more ingredient(s) selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant and a surfactant.

The pharmaceutical, cosmetic or cosmeceutical compositions of the present invention have a pH value that preferably ranges between about 4.0 and about 7.0, more preferably between about 5.0 and about 6.0.

The pharmaceutical, cosmetic or cosmeceutical compositions of the present invention are preferably devoid of an enduring perfume composition.

According to yet another aspect of the present invention there are provided processes of preparing the pharmaceutical, cosmetic or cosmeceutical compositions of the present invention. Each of the processes comprises admixing the urea and/or the derivative thereof, the propellant(s) and a pharmaceutically, cosmetically or cosmeceutically acceptable carrier.

According to further features in preferred embodiments of the invention described below, in cases where the composition further comprises any of the additional active ingredients or ingredients described hereinabove, the processes further comprise admixing the active ingredient or any other ingredient with the urea and/or the derivative thereof, the propellant(s) and the carrier.

According to still another aspect of the present invention there are provided methods of treating a medical, cosmetic and/or a cosmeceutical condition. The methods comprise topically applying onto one or more biological surface(s) of a subject in need thereof, a pharmaceutically, cosmetically or cosmeceutically effective amount of the composition described hereinabove.

According to still further features in the described preferred embodiments the biological surface(s) is selected from the group consisting of a lateral aspect of a forearm, a lateral aspect of a leg, an elbow, a palm, a foot, a backhand, a back and a scalp.

The present invention successfully addresses the shortcomings of the presently known configurations by providing foamable compositions containing urea and/or a derivative thereof and methods of treating various dermatological conditions such as conditions associated with dry skin and/or scalp utilizing same, which are highly efficient and convenient, and are thus superior to the presently available urea preparations.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### DESCRIPTION OF THE PREFERRED EMBODIMENTS

The present invention is of compositions for topical application which can be efficiently used in the treatment of various medical, cosmetic and/or cosmeceutical conditions. Specifically, the present invention is of (i) compositions for topical application, which contain, as an active ingredient urea and/or a derivative thereof; (ii) processes of preparing these compositions; and (iii) their use in treating medical, cosmetic and/or cosmeceutical conditions associated with dry skin and/or scalp such as, but not limited to, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruff and xeroderma pigmentosum, as well as other dermatological conditions.

The principles and operation of the compositions, processes and methods according to the present invention may be better understood with reference to the Examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed in detail hereinabove, urea is known as an efficient hydrating agent, which therefore serves as a potent agent for treating dry skin conditions. The hydrating efficacy and hence, the therapeutic or cosmetic performance, as well as the usage convenience of hydrating agents and any other agents for topical application depend, inter alia, on the final form of the composition in which these agents are formulated.

As is further discussed in detail in the Background section above, foam formulations are well suited for the topical application of compositions. However, the presently known preparations that include urea as the active ingredient are typically formulated as creams, lotions, gels, ointment and the like, and therefore their efficacy and usage convenience are limited. On the other hand, although foam compositions which include urea as an optional ingredient are known, these compositions are not aimed at treating dry skin or scalp conditions and therefore do not include urea in a substantial concentration (e.g., higher than 5 weight percentages), such that it may serve as the main active hydrating agent.

In a search for an efficient composition for treating dry skin and scalp, as well as other medical, cosmetic and cosmeceutical conditions, which would overcome the disadvantages of the presently known formulations, the present inventors have surprisingly found that a composition that comprises urea, preferably in a relatively high concentration, which is formulated as a foam, is highly efficient in the treatment of dry skin and/or scalp and is further characterized by improved absorption, after feel and comfort, as compared with the presently known formulations, and is devoid of stickiness and other adverse effects that accompany the use of the presently known formulations. As is discussed in detail hereinabove, due to its solid-like nature, the topical application of such a foamable composition is highly efficient and convenient, as compared with the presently known urea formulations.

Hence, according to one aspect of the present invention, there is provided a foamable pharmaceutical, cosmetic or cosmeceutical composition for topical application, which is identified for use in the treatment of a medical, cosmetic and/or cosmeceutical condition associated with dry skin and/or scalp. The composition, according to this aspect of the present invention, comprises urea, one or more propellant(s) and a pharmaceutically, cosmetically or cosmeceutically acceptable carrier.

As used herein, the phrase “topical application” describes application onto a biological surface, e.g., skin or scalp. Hence, the phrase “a composition for topical application” describes a composition that is applied to a subject by direct laying or spreading on the skin, scalp or any other biological surface of the subject.

As the composition of the present invention is aimed at treating dry skin and/or scalp conditions, the topical application is preferably performed onto a dry skin area. The dry skin area can be one or more of, for example, the lateral aspect of a forearm, the lateral aspect of a leg, an elbow, a palm, a foot, a backhand, a back and/or a scalp.

As used herein throughout the term “comprising” means that other steps and ingredients which do not affect the end results can be added. This term encompasses the terms “consisting of” and “consisting essentially of”.

The phrase “consisting essentially of” means that the composition may include additional ingredients, but only if the additional ingredients do not materially alter the basic and novel characteristics of the claimed compositions or methods.

The phrase “active ingredient” as used herein means an ingredient that exerts a pharmaceutical, cosmetic or cosmeceutical activity. As urea is a known hydrating agent and thus the composition of the present invention is preferably directed to treat or prevent dry skin or scalp, the phrase “active ingredient” whenever used herein in the context of urea and/or a related substance refers to an ingredient that exerts hydration activity, namely, a hydrating agent. While urea is a well known and widely used hydrating agent, derivatives of urea are also known to exert hydration properties, as is described, for example, in EP Application No. 0101887 A2. Such urea derivatives can therefore be beneficially used in the composition of the present invention, in addition to or instead of urea.

Hence, according to an embodiment of the present invention, the pharmaceutical, cosmetic or cosmeceutical composition for topical application comprises urea and/or a derivative thereof.

An urea derivative, according to the present invention, can be described by the general formula:
R¹R²N—C(═O)—NR³R⁴,
wherein each of R¹, R², R³ and R⁴ is independently selected from the group consisting of hydrogen, alkyl, cycloalkyl, alkenyl and aryl, or, alternatively, one of R¹ and R² and one of R³ and R⁴ are covalently linked therebetween to thereby form a heteroalicyclic ring.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it means that the group, in this case the alkyl group, may contain 1 carbon atom, 2 carbon atoms, 3 carbon atoms, etc., up to and including 20 carbon atoms. More preferably, it is a medium size alkyl having 1 to 10 carbon atoms. Most preferably, it is a lower alkyl having 1 to 4 carbon atoms. The alkyl group may be substituted or unsubstituted. When substituted, the substituent group can be, for example, hydroxy, halo, amino, nitro, cyano, alkoxy, aryloxy, thiohydroxy, thioalkoxy, thioaryloxy, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, hydroxy, halo, amino, nitro, cyano, alkoxy, aryloxy, thiohydroxy, thioalkoxy, thioaryloxy, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

An “alkenyl” group refers to an alkyl group, as is defined hereinabove, which consists of at least two carbon atoms and at least one carbon-carbon double bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, hydroxy, halo, amino, nitro, cyano, alkoxy, aryloxy, thiohydroxy, thioalkoxy, thioaryloxy, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

A “hydroxy” group refers to an —OH group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R′ group, where R′ is as defined herein for R′.

A “C-carboxy” group refers to a —C(═O)—O—R′ groups, where R′ is as defined herein.

An “O-carboxy” group refers to an R′C(═O)—O— group, where R′ is as defined herein.

A “halo” group refers to fluorine, chlorine, bromine or iodine.

A “trihalomethyl” group refers to a —CX₃ group wherein X is a halo group as defined herein.

A “sulfinyl” group refers to a —S(═O)—R′ group, where R′ is as defined herein.

A “sulfonyl” group refers to a —S(═O)₂—R′ group, where R′ is as defined herein.

A “S-sulfonamido” group refers to a —S(═O)₂—NR′R″ group, with R′ is as defined herein and R″ is as defined herein for R′.

A “N-sulfonamido” group refers to an R′S(═O)₂—NR″ group, where R′ and R″ are as defined herein.

An “Amino” group refers to an —NR′R″ group where R′ and R″ are as defined herein.

A “C-amido” group refers to a —C(═O)—NR′R″ group, where R′ and R″ are as defined herein.

A “N-amido” group refers to an R′C(═O)—NR″ group, where R′ and R″ are as defined herein.

A “nitro” group refers to a —NO₂ group.

A “cyano” group refers to a —C≡N group.

The term “phosphonyl” describes an —O—P(═O)(OR′)(OR″) group, with R′ and R″ as defined hereinabove.

The term “phosphinyl” describes a —PR′R″ group, with R′ and R″ as defined hereinabove.

As urea, and/or a derivative thereof, serves as the main active ingredient in the composition of the present invention, its concentration is relatively high, so as to efficiently exert a hydrating effect.

Thus, the concentration of the urea and/or the derivative thereof in the composition of the present invention is preferably greater than 5 weight percentages, more preferably greater than 6 weight percentages, more preferably greater than 7 weight percentages, more preferably greater than 8 weight percentages, more preferably greater than 9 weight percentages, more preferably greater than 10 weight percentages, more preferably greater than 11 weight percentages, more preferably greater than 12 weight percentages, more preferably greater than 13 weight percentages, more preferably greater than 14 weight percentages, more preferably greater than 15 weight percentages, more preferably greater than 16 weight percentages, more preferably greater than 17 weight percentages, more preferably greater than 18 weight percentages, more preferably greater than 19 weight percentages and, according to one of the presently most preferred embodiments of the present invention, it is about 20 weight percentages.

However, the concentration of the urea and/or the derivative thereof in the composition of the present invention can further preferably be greater than 20 weight percentages, more preferably greater than 21 weight percentages, more preferably greater than 22 weight percentages, more preferably greater than 23 weight percentages, more preferably greater than 24 weight percentages, more preferably greater than 25 weight percentages, more preferably greater than 26 weight percentages, more preferably greater than 27 weight percentages, more preferably greater than 28 weight percentages, more preferably greater than 29 weight percentages, more preferably greater than 30 weight percentages, more preferably greater than 31 weight percentages, more preferably greater than 32 weight percentages, more preferably greater than 33 weight percentages, more preferably greater than 34 weight percentages, more preferably greater than 35 weight percentages, more preferably greater than 36 weight percentages, more preferably greater than 37 weight percentages, more preferably greater than 38 weight percentages, more preferably greater than 39 weight percentages, and according to one of the presently most preferred embodiments of the present invention, it is about 40 weight percentages. The concentration of the urea and/or the derivative thereof in the composition of the present invention can further preferably be greater than 40 weight percentages and up to about 48 weight percentages.

The phrase “greater than” as used herein with respect to a numerical indication (e.g., a concentration) encompasses any number (integral or fractional) that is greater than the indicated number.

Hence, the concentration of the urea and/or the derivative thereof in the composition preferably ranges between, for example, about 5.1 weight percentages and about 48 weight percentages, more preferably between about 10 and about 40 weight percentages, and even more preferably between about 20 and about 40 weight percentages.

As used herein throughout, the phrase “weight percentages” describes the weight percentages (of an ingredient) of the total weight of a composition containing same.

As used herein the term “about” refers to ±10%.

The composition of the present invention further includes a pharmaceutically, cosmetically or cosmeceutically acceptable carrier.

As used herein, the term “pharmaceutically, cosmetically or cosmeceutically acceptable carrier” describes a carrier or a diluent that does not cause significant irritation to an organism and does not abrogate the biological activity and properties of the applied active ingredient.

Examples of acceptable carriers that are usable in the context of the present invention include carrier materials that are well-known for use in the cosmetic and medical arts as bases for foams and the like.

The composition of the present invention is formulated in the form of a foam. Preferably, the foam is formed by the passage of a pressurized mixture of a concentrate and a propellant through a nozzle. Preferably, the propellant is in the form of a compressed gas, typically a liquefiable gas. The mixture is preferably contained in a dispenser equipped with a dispensing head and valve, and pressurized with the propellant. Upon discharge of the composition through the dispensing head, the volatilization of the dispersed liquid droplets of propellant causes the dispensed concentrate to foam. Depending upon the precise formulation of the concentrate and the propellant, the dispensed product may range from a dense creamy foam to a light foam, dependent on desired aesthetics in the hand and when spread onto the substrate.

The concentration of the propellant in the composition preferably ranges between about 0.5 and about 60 weight percentages, more preferably between about 1 and about 20 weight percentages of the total composition.

Any propellant suitable for use in pharmaceutical, cosmetic or cosmeceutical compositions can be used herein, alone or in combination with other propellant. Non-limiting examples of suitable propellants include nitrous oxide, carbon dioxide, nitrogen, and hydrocarbon propellants such as propane, iso-butane, n-butane, isopentane, n-pentane, and dimethyl ether. Preferred propellants are selected from, for example, propane, iso-butane, n-butane, isopentane, n-pentane, and mixtures thereof. Chlorinated fluorocarbons such as 1,1-difluoro- or 1,1,1,2-tetrafluoroethane are also suitable but their use is being limited for environmental reasons. The propellants listed above typically have a low boiling point and are in a gaseous form at room temperature in standard conditions.

The composition of the present invention can optionally further comprise a variety of components that are suitable for rendering the composition more cosmetically or aesthetically acceptable or to provide the composition with additional usage benefits. Such conventional optional components or ingredients are well known to those skilled in the art and are referred to herein as “ingredients”. These include any cosmetically acceptable ingredients such as those found in the CTFA International Cosmetic Ingredient Dictionary and Handbook, 8th edition, edited by Wenninger and Canterbery, (The Cosmetic, Toiletry, and Fragrance Association, Inc., Washington, D.C., 2000). Some non-limiting representative examples of these ingredients include humectants, deodorants, antiperspirants, sun screening agents, sunless tanning agents, hair conditioning agents, pH adjusting agents, chelating agents, preservatives, emulsifiers, occlusive agents, emollients, thickeners, solubilizing agents, penetration enhancers, anti-irritants, colorants and surfactants.

Thus, the composition of the present invention can comprise, in combination with urea and/or a derivative thereof, one or more additional humectants or moisturizing agents. Representative examples of humectants that are usable in this context of the present invention include, without limitation, guanidine, glycolic acid and glycolate salts (e.g. ammonium salt and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

The composition of the present invention can further comprise a pH-adjusting agent. Suitable pH adjusting agents include, for example, one or more adipic acids, glycines, citric acids, calcium hydroxides, magnesium aluminometasilicates, buffers or any combinations thereof.

As is widely recognizable in the art, since the skin pH is 5.5, compositions for topical application should preferably have a pH value of between 4.0 and 7.0, preferably between 5.0 and 6.0, most preferably about 5.5 or substantially 5.5, so as to avoid irritation. Hence, a pH adjusting agent is typically added so as to bring the pH of the composition to the desired value. The composition of the present invention is therefore preferably formulated so as to have a pH value that ranges between about 4.0 and about 7.0, more preferably between about 5.0 and about 6.0.

Representative examples of deodorant agents that are usable in the context of the present invention include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmlthyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the composition of the present invention either in a solubilized or a particulate form and include, for example, aluminum or zirconium astringent salts or complexes.

Representative examples of sun screening agents usable in context of the present invention include, without limitation, p-aminobenzoic acid, salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one) and 4-isopropyl-di-benzoylmethane, and any combination thereof.

Representative examples of sunless tanning agents usable in context of the present invention include, without limitation, dihydroxyacetone, glyceraldehyde, indoles and their derivatives. The sunless tanning agents can be used in combination with the sunscreen agents.

Suitable hair conditioning agents that can be used in the context of the present invention include, for example, one or more collagens, cationic surfactants, modified silicones, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof.

The chelating agents are optionally added to the composition of the present invention so as to enhance the preservative or preservative system. Preferred chelating agents are mild agents, such as, for example, ethylenediaminetetraacetic acid (EDTA), EDTA derivatives, or any combination thereof.

Suitable preservatives for use in the composition of the present composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

Suitable emulsifiers that can be used in the context of the present invention include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents that can be used in the context of the present invention include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients, other than urea and a derivative thereof, that can be used in the context of the present invention include, for example, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners that can be used in the context of the present invention include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, fatty acids and their alkali salts and mixtures thereof.

Representative examples of solubilizing agents that are usable in this context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEENS and spans, e.g., TWEEN 80. Other solubilizers that are usable for the composition of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

Suitable penetration enhancers usable in context of the present invention include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, and the like. The permeation enhancer may also be a vegetable oil. Such oils include, for example, safflower oil, cottonseed oil and corn oil.

Suitable anti-irritants that can be used in the context of the present invention include, for example, steroidal and non steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

Although a wide variety of ingredients can be included in the composition of the present invention, in addition to the active ingredients, the composition is preferably devoid of an enduring perfume composition. The incorporation of such a perfume composition in pharmaceutical compositions is considered in the art disadvantageous for skin and scalp medical treatment, as it oftentimes cause undesirable irritation of a sensitive skin.

As used herein, the phrase “an enduring perfume composition” describes a composition that comprises one or more perfumes that provide a long lasting aesthetic benefit with a minimum amount of material. Enduring perfume compositions are substantially deposited and remain on the body throughout any rinse and/or drying steps. Representative examples of such compositions are described, for example, in U.S. Pat. No. 6,086,903.

However, it should be noted that fragrances other than enduring perfume compositions, perfumes or perfume compositions, which are fast removable from the surface they are deposited on, can be included in the composition of the present invention.

Further optionally, the composition of the present invention can comprise one or more additional active ingredients, which are aimed at providing the composition with an additional therapeutic, cosmeceutical or cosmetic effect.

As is described hereinabove, the term “active ingredient” refers to an ingredient which exerts a pharmacological, dermatological or any other beneficial activity. An “additional active ingredient” refers herein to any active ingredient other than urea or derivatives thereof, as is described hereinabove.

Compositions according to the present invention, which further comprises one or more additional active ingredients, can therefore be further efficiently used, in addition to treatment of a condition associated with dry skin and/or scalp, in the treatment of any medical, cosmetic and/or cosmeceutical condition in which applying the additional active ingredient is beneficial.

Preferred additional active ingredients according to the present invention include, without limitation, one or more, or any combination of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an anti-dandruff agent.

Suitable anti-acne agents for use in this context of the present invention include, without limitation, keratolytics such as salicylic acid, sulfur, glycolic, pyruvic acid, resorcinol, and N-acetylcysteine and retinoids such as retinoic acid and its derivatives (e.g., cis and trans, esters).

Suitable antibiotics for use in this context of the present invention include, without limitation, benzoyl peroxide, octopirox, erythromycin, zinc, tetracyclin, triclosan, azelaic acid and its derivatives, phenoxy ethanol and phenoxy proponol, ethylacetate, clindamycin and meclocycline; sebostats such as flavinoids; alpha and beta hydroxy acids; and bile salts such as scymnol sulfate and its derivatives, deoxycholate and cholate.

Representative examples of non-steroidal anti-inflammatory agents that are usable in this context of the present invention include, without limitation, oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical application.

Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Suitable antipruritic agents for use in this context of the present invention include, without limitation, pharmaceutically acceptable salts of methdilazine and trimeprazine.

Non-limiting examples of anesthetic drugs that are suitable for use in the context of the present invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Suitable antimicrobial agents, including antibacterial, antifungal, antiprotozoal and antiviral agents, for use in the context of the present invention include, without limitation, beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole and mixtures thereof.

Non-limiting examples of suitable anti-oxidants for use in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of suitable chemotherapeutic agents for use in the context of the present invention include daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

Non-limiting examples of suitable antidepressants for use in the context of the present invention include norepinephrine-reuptake inhibitors (“NRIs”), selective-serotonin-reuptake inhibitors (SSRIs), monoamine-oxidase inhibitors (MAOIs), serotonin-and-noradrenaline-reuptake inhibitors (“SNFIs), corticotropin-releasing factor (CRF) antagonists, α-adrenoreceptor antagonists, NK1-receptor antagonists, 5-HT_{1A}-receptor agonist, antagonists, and partial agonists and atypical antidepressants, as well as norepinephrine-reuptake inhibitors such as, but are not limited to amitriptyline, desmethylamitriptyline, clomipramine, doxepin, imipramine, imipramine-oxide, trimipramine; adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, and serotonin-reuptake inhibitors such as, but are not limited to, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine.

Exemplary anti-dandruff ingredients usable in the context of the present invention include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, and ciclopirox olamine, and mixtures thereof.

Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

Non-limiting examples of dermatological active ingredients usable in context of the present invention include jojoba oil and aromatic oils such as methyl salicylate, wintergreen, peppermint oil, bay oil, eucalyptus oil and citrus oils, as well as ammonium phenolsulfonate, bismuth subgallate, zinc phenolsulfonate and zinc salicylate. Non-limiting examples of antifungal agents include miconazole, clotrimazole, butoconazole, fenticonasole, tioconazole, terconazole, sulconazole, fluconazole, haloprogin, ketonazole, ketoconazole, oxinazole, econazole, itraconazole, terbinafine, nystatin and griseofulvin.

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

Suitable hormones for use in the context of the present invention include, for example, androgenic compounds and progestin compounds.

Representative examples of androgenic compounds include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any of the foregoing.

Representative examples of progestin compounds include, without limitation, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and mixtures thereof.

The composition of the present invention may be packed or presented in any convenient way. For example, they may be packed in a tube, a bottle, or a pressurized container, using techniques well known to those skilled in the art and as set forth in reference works such as Remington's Pharmaceutical Science 15^{th} Ed. It is preferred that the packaging is done in such a way so as to minimize contact of the unused compositions with the environment, in order to minimize contamination of the composition before and after the container is opened.

As the composition of the present invention includes urea and/or a derivative thereof, preferably in a substantial concentration, it is highly beneficial in preventing or treating medical, cosmetic and/or cosmeceutical conditions associated with dry skin and/or scalp such as, for example, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

Hence, in a preferred embodiment of the present invention, the composition described hereinabove, is packaged in a packaging material and is identified in print, in or on the package, for use in the treatment or prevention of dry skin and/or scalp and/or any one or more of the conditions described or listed herein.

Further, according to another aspect of the present invention, there is provided a method of treating a medical, cosmetic and/or cosmeceutical condition. The method is effected by topically applying onto one or more affected biological surface(s) of a subject in need thereof, e.g. dry skin and/or scalp, a pharmaceutically, cosmetically or cosmeceutically effective amount of the composition of the present invention as described herein.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “topically applying” describes application onto one or more biological surface(s), e.g., skin or scalp, by direct laying or spreading a composition on the surface. Non-limiting examples of biological surfaces onto which the composition of the present invention can be topically applied include the lateral aspect of forearms, the lateral aspect of legs, elbows, palms, feet, backhands, back, scalp and any other dry skin surface.

A representative, non-limiting example of an application regime of the composition of the present invention, according to this aspect of the present invention, includes topical application of the composition between one and four times a day, more preferably twice a day (e.g., once in the morning and once in the evening). The topical application of the composition of the present invention is preferably carried out for a time period that ranges between 1 and 30 days, more preferably for a time period of about fourteen days.

The phrase “pharmaceutically, cosmetically or cosmeceutically effective amount” describes an amount of a composition that is sufficient to significantly induce a positive modification in the condition being treated, but low enough to avoid significant side effects, within the scope of sound judgment of the skilled artisan. The effective amount of the composition may vary with the particular skin being treated, the age and physical condition of the biological subject being treated, the severity of the condition, the duration of the treatment, the nature of concurrent therapy, the specific compound, composition or other material employed, the particular pharmaceutically, cosmetically or cosmeceutically acceptable topical carrier utilized, and like factors within the knowledge and expertise of the skilled artisan.

While the method according to this aspect of the present invention is preferably beneficial in treating conditions associated with dry skin and/or scalp, in cases where the compositions of the present invention further comprises additional active ingredient(s), the method can be further used for treating other conditions in which applying the additional active ingredient(s) is beneficial. Such conditions include, for example, infections, fungi, allergies, aging and more.

According to another aspect of the present invention there is provided a process of preparing the novel composition described hereinabove. The process generally comprises admixing the urea and/or the derivative thereof, as described hereinabove, the propellant(s) and the pharmaceutically, cosmetically or cosmeceutically acceptable carrier. In cases were other ingredients or active ingredient, as is detailed hereinabove, are present in the composition, the process includes admixing these ingredients together with the urea and/or the derivative thereof, the propellant(s) and the carrier. The mixing technique utilized in the process of the present invention can be any one of the known techniques for formulating foamable compositions. A variety of exemplary formulation techniques that are usable in the process of the present invention is described, for example, in Harry's Cosmeticology, Seventh Edition, Edited by J B Wilkinson and R J Moore, Longmann Scientific & Technical, 1982, Chapter 13 “The Manufacture of Cosmetics” pages 757-799.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

A representative example of a foam skin and scalp topical composition according to the present invention, containing urea in a 40 weight percentages concentration, was prepared using conventional methods (see, for example, Harry's Cosmeticology, Seventh Edition, Edited by J B Wilkinson and R J Moore, Longmann Scientific & Technical, 1982, Chapter 13 “The Manufacture of Cosmetics” pages 757-799). The composition comprises about 10 weight percentages of a propellant, as described hereinabove. Other components of the composition are listed in Table 1 hereinbelow.

TABLE 1

| | |
|---|---|
| GLYCERINE | 2.0 |
| ALLANTOIN | 0.2 |
| UREA USP | 40.0 |
| CETYL ALCOHOL | 0.5 |
| VASELINE ™ | 5.0 |
| POLYSILANE | 3.0 |
| MYRITOL 318 ™ | 2.0 |
| TWEEN 60 ™ | 1.0 |
| SILICON D.C.350 ™ | 0.5 |
| VIT. E ACETATE | 0.1 |
| MONTANOV 68 ™ | 1.5 |
| PHENONIP ™ | 0.7 |
| AMYLUN RICE STARCH ™ | 2.0 |
| DMDM HYDANTOIN ™ | 0.35 |
| WATER | q.s |

### Example 2

Another representative example of a foam skin and scalp topical composition according to the present invention, containing urea in a 20 weight percentages concentration, was prepared using conventional methods (see, for example, Harry's Cosmeticology, Seventh Edition, Edited by J B Wilkinson and R J Moore, Longmann Scientific & Technical, 1982, Chapter 13 “The Manufacture of Cosmetics” pages 757-799). The composition comprises about 10 weight percentages of a propellant, as described hereinabove. Other components of the composition are listed in Table 2 hereinbelow.

TABLE 2

| | |
|---|---|
| GLYCERINE | 2.0 |
| ALLANTOIN | 0.2 |
| UREA USP | 20.0 |
| CETYL ALCOHOL | 0.5 |
| VASELINE ™ | 8.0 |
| ISOPROPYL MYRISTATE | 4.0 |
| MYRITOL 318 ™ | 3.0 |
| TWEEN 60 ™ | 1.0 |
| SILICON D.C.350 ™ | 0.5 |
| VIT. E ACETATE | 0.1 |
| MONTANOV 68 ™ | 1.5 |
| PHENONIP ™ | 0.7 |
| AMYLUN RICE STARCH ™ | 2.0 |
| DMDM HYDANTOIN ™ | 0.35 |
| WATER | q.s |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A foamable pharmaceutical, cosmetic or cosmeceutical composition for topical application, identified for use in the treatment of a medical, cosmetic and/or cosmeceutical condition associated with dry skin and/or scalp, comprising urea and/or a derivative thereof, at least one propellant and a pharmaceutically, cosmetically or cosmeceutically acceptable carrier.

2. The foamable pharmaceutical, cosmetic or cosmeceutical composition of claim 1, packaged in a packaging material and identified in print, in or on said packaging material, for use in the treatment of said condition.

3. The foamable pharmaceutical, cosmetic or cosmeceutical composition of claim 1, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruff and xeroderma pigmentosum.

4. The foamable pharmaceutical, cosmetic or cosmeceutical composition of claim 2, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

5. The foamable pharmaceutical, cosmetic or cosmeceutical composition of claim 1, wherein a concentration of said urea and/or said derivative thereof is greater than 5 weight percentages of the composition.

6. The foamable pharmaceutical, cosmetic or cosmeceutical composition of claim 5, wherein said concentration of said urea and/or said derivative thereof is greater than 10 weight percentages of the composition.

7. The foamable pharmaceutical, cosmetic or cosmeceutical composition of claim 5, wherein said concentration of said urea and/or said derivative thereof ranges between 5.1 weight percentages and about 48 weight percentages of the composition.

8. The foamable pharmaceutical, cosmetic or cosmeceutical composition of claim 7, wherein said concentration of said urea and/or said derivative thereof ranges between about 20 weight percentages and about 48 weight percentages of the composition.

9. The foamable pharmaceutical, cosmetic or cosmeceutical composition of claim 1, wherein a concentration of said at least one propellant ranges between about 0.5 weight percentage and about 60 weight percentages.

10. The foamable pharmaceutical, cosmetic or cosmeceutical composition of claim 9, wherein said concentration of said at least one propellant ranges between about 10 weight percentages and about 20 weight percentages.

11. The foamable pharmaceutical, cosmetic or cosmeceutical composition of claim 1, wherein said at least one propellant is selected from the group consisting of propane, iso-butane, n-butane, isopentane, n-pentane, and mixtures thereof.

12. The foamable pharmaceutical, cosmetic or cosmeceutical composition of claim 1, being devoid of an enduring perfume composition.

13. The foamable pharmaceutical, cosmetic or cosmeceutical composition of claim 1, further comprising at least one additional active ingredient.

14. The foamable pharmaceutical, cosmetic or cosmeceutical composition of claim 13, wherein said at least one additional active ingredient is selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

15. The foamable pharmaceutical, cosmetic or cosmeceutical composition of claim 13, further identified for use in the treatment of a medical, cosmetic and/or cosmeceutical condition in which applying said at least one additional active ingredient is beneficial.

16. The foamable pharmaceutical, cosmetic or cosmeceutical composition of claim 1, further comprising at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant and a surfactant.

17. The foamable pharmaceutical, cosmetic or cosmeceutical composition of claim 1, having a pH value that ranges between about 4.0 and about 7.0.

18. The foamable pharmaceutical, cosmetic or cosmeceutical composition of claim 17, having a pH value that ranges between about 5.0 and about 6.0.

19. A process of preparing a foamable pharmaceutical, cosmetic or cosmeceutical composition for topical application, identified for use in the treatment of a medical, cosmetic and/or cosmeceutical condition associated with dry skin and/or scalp, the process comprising admixing urea and/or a derivative thereof, at least one propellant and a pharmaceutically, cosmetically or cosmeceutically acceptable carrier.

20. The process of claim 19, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

21. The process of claim 19, wherein a concentration of said urea and/or said derivative thereof is greater than 5 weight percentages of said composition.

22. The process of claim 21, wherein said concentration of said urea and/or said derivative thereof is greater than 10 weight percentages of said composition.

23. The process of claim 21, wherein said concentration of said urea and/or said derivative thereof ranges between 5.1 weight percentages and about 48 weight percentages of said composition.

24. The process of claim 23, wherein said concentration of said urea and/or said derivative thereof ranges between about 20 weight percentages and about 48 weight percentages of said composition.

25. The process of claim 19, wherein a concentration of said at least one propellant ranges between about 0.5 weight percentage and about 60 weight percentages.

26. The process of claim 25, wherein said concentration of said at least one propellant ranges between about 10 weight percentages and about 20 weight percentages.

27. The process of claim 19, wherein said at least one propellant is selected from the group consisting of propane, iso-butane, n-butane, isopentane, n-pentane, and mixtures thereof.

28. The process of claim 19, further comprising admixing, with said urea and/or said derivative thereof, said at least one propellant and said carrier, at least one additional active ingredient.

29. The process of claim 28, wherein said at least one additional active ingredient is selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

30. The process of claim 19, further comprising admixing, with said urea and/or said derivative thereof, said at least one propellant and said carrier, at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant and a surfactant.

31. The process of claim 19, wherein said composition has a pH value that ranges between about 4.0 and about 7.0.

32. The process of claim 31, wherein said composition has a pH value that ranges between about 5.0 and about 6.0.

33. The process of claim 19, wherein said composition is devoid of an enduring perfume composition.

34. A method of treating a medical, cosmetic and/or a cosmeceutical condition, the method comprising topically applying, onto at least one biological surface of a subject in need thereof, a pharmaceutically, cosmetically or cosmeceutically effective amount of the foamable pharmaceutical, cosmetic or cosmeceutical composition of claim 1.

35. The method of claim 34, wherein said medical, cosmetic and/or cosmeceutical condition is a condition associated with dry skin and/or scalp.

36. The method of claim 35, wherein said condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

37. The method of claim 34, wherein said at least one biological surface is selected from the group consisting of a lateral aspect of a forearm, a lateral aspect of a leg, an elbow, a palm, a foot, a backhand, a back and a scalp.

38. The method of claim 34, wherein a concentration of said urea and/or said derivative thereof is greater than 5 weight percentages of said composition.

39. The method of claim 38, wherein said concentration of said urea and/or said derivative thereof is greater than 10 weight percentages of said composition.

40. The method of claim 38, wherein said concentration of said urea and/or said derivative thereof ranges between 5.1 weight percentages and about 48 weight percentages of said composition.

41. The method of claim 40, wherein said concentration of said urea and/or said derivative thereof ranges between about 20 weight percentages and about 48 weight percentages of said composition.

42. The method of claim 34, wherein a concentration of said at least one propellant ranges between about 0.5 weight percentage and about 60 weight percentages.

43. The method of claim 42, wherein said concentration of said at least one propellant ranges between about 10 weight percentages and about 20 weight percentages.

44. The method of claim 34, wherein said at least one propellant is selected from the group consisting of propane, iso-butane, n-butane, isopentane, n-pentane, and mixtures thereof.

45. The method of claim 34, wherein said composition is devoid of an enduring perfume composition.

46. The method of claim 34, wherein said composition further comprises at least one additional active ingredient.

47. The method of claim 46, wherein said at least one additional active ingredient is selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

48. The method of claim 46, wherein said medical, cosmetic and/or cosmeceutical condition is a condition in which applying said at least additional active ingredient is beneficial.

49. The method of claim 34, wherein said composition further comprises at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant and a surfactant.

50. The method of claim 34, wherein said composition has a pH value that ranges between about 4.0 and about 7.0.

51. The method of claim 50, wherein said composition has a pH value that ranges between about 5.0 and about 6.0.
